(19) United States
(12) Patent Application Publication
(10) Pub. No.: US 2026/0212697 A1
(43) Pub. Date: Jul. 23, 2026

(54) DISPLAY DEVICE AND CONTROL METHOD THEREOF

(71) Applicant: SAMSUNG ELECTRONICS CO., LTD., Suwon-si (KR)

(72) Inventors: Donghoon LEE, Suwon-si (KR); Dongwon Kim, Suwon-si (KR); Sangshin Park, Suwon-si (KR); Eunae Cho, Suwon-si (KR); Yunjoo Cho, Suwon-si (KR)

(73) Assignee: SAMSUNG ELECTRONICS CO., LTD., Suwon-si (KR)

(21) Appl. No.: 19/645,937

(22) Filed: Apr. 13, 2026

Related U.S. Application Data
(63) Continuation of application No. PCT/KR2024/014702, filed on Sep. 27, 2024.

(30) Foreign Application Priority Data
Oct. 13, 2023 (KR) 10-2023-0137012

(51) Int. Cl. G06V 40/10; A61B 5/00; A61B 5/103; G06V 10/14; G06V 10/36; G06V 10/98; G06V 40/16

(52) U.S. Cl. CPC G06V 40/15; A61B 5/0077; A61B 5/1032; A61B 5/7278; A61B 5/743; G06V 10/14; G06V 10/36; G06V 10/98; G06V 40/161

(57) ABSTRACT

A display apparatus and method are provided for identifying a biometric signal of a user while content is displayed. The display apparatus includes a camera, a display, a memory, and at least one processor. While content is output through the display, the processor obtains an image through the camera in response to an occurrence of a preset event. The processor determines whether to perform filtering on the obtained image based on a plurality of first pixel values corresponding to a facial area of a user identified in the obtained image and a plurality of second pixel values corresponding to the content displayed through the display. When filtering is determined to be performed, the processor generates a corrected image by filtering the obtained image based on the plurality of second pixel values. The processor then identifies a biometric signal of the user based on the corrected image.

### CROSS-REFERENCE TO RELATED APPLICATIONS

This application is a bypass continuation application of International Application No. PCT/KR2024/014702, filed on Sep. 27, 2024, which claims priority to Korean Patent Application No. 10-2023-0137012, filed on Oct. 13, 2023, in the Ministry Intellectual Property, the disclosures of which are incorporated by reference herein in their entireties.

### BACKGROUND

### 1. Field

The disclosure relates to a display apparatus and a control method thereof. More particularly, the disclosure relates to a display apparatus that obtains a biometric signal of a user that uses the display apparatus and a control method thereof.

### 2. Description of Related Art

As interest on home care increases, use of a so-called health care electronic apparatus that obtains biometric information of a user and manages the user's health has increased. Through a health care apparatus, the user may be provided with information about a health state of the user in real-time without having to visit with a professional and a professional medical institution.

Specifically, with recent developments in electronic technology, users limited to only health care electronic apparatuses are now able to receive home care service from general electronic apparatuses. Specifically, electronic apparatuses that include a camera at one surface thereof such as a notebook, a monitor, a smart phone, and the like may obtain an image of a user through the camera. At this time, the electronic apparatus may obtain biometric information of the user by analyzing the obtained image, generate information about the health state of the user based on the obtained biometric information and provide to the user.

### SUMMARY

According to an aspect of the disclosure, a display apparatus includes a camera; a display; memory storing one or more instructions; and at least one processor configured to execute the one or more instructions. The one or more instructions, when executed by the at least one processor individually or collectively, cause the display apparatus to: based on an occurrence of a pre-set event, obtain an image through the camera while content is being output through the display, determine, based on a plurality of first pixel values corresponding to a facial area of a user identified in the obtained image and a plurality of second pixel values corresponding to the content, whether to perform filtering on the obtained image, based on a determination that filtering is to be performed on the obtained image, obtain a corrected image by performing filtering on the obtained image based on the plurality of second pixel values, and identify a biometric signal of the user based on the corrected image.

The one or more instructions, when executed by the at least one processor individually or collectively, cause the display apparatus to: determine to perform filtering on the obtained image based on a correlation degree between the plurality of first pixel values and the plurality of second pixel values being greater than or equal to a pre-set first value.

The one or more instructions, when executed by the at least one processor individually or collectively, cause the display apparatus to: identify, based on the plurality of first pixel values corresponding to the facial area of the user and a plurality of third pixel values corresponding to a remaining area other than the facial area of the user identified from the obtained image, a contrast ratio between the facial area and the remaining area, and determine to perform filtering on the obtained image based on the identified contrast ratio being greater than or equal to a pre-set second value.

The one or more instructions, when executed by the at least one processor individually or collectively, cause the display apparatus to: determine to perform filtering on the obtained image based on an average value of the plurality of second pixel values being greater than or equal to a pre-set third value, the plurality of second pixel values corresponding to a green channel among a plurality of channels of the content.

The one or more instructions, when executed by the at least one processor individually or collectively, cause the display apparatus to: for each image frame of a plurality of image frames of the content, identify an average value of the plurality of second pixel values corresponding to a green channel of the image frame, identify, based on each average value corresponding to each image frame, a frequency corresponding to a green channel of the content, and determine to perform filtering on the obtained image based on the identified frequency being within a pre-set range and each average value of each frame of the plurality of image frames of the content being greater than or equal to a pre-set third value.

The one or more instructions, when executed by the at least one processor individually or collectively, cause the display apparatus to: apply a first weight value corresponding to a luminance setting value of the display apparatus to the plurality of second pixel values, and perform, based on a plurality of fourth pixel values corresponding to the obtained image and the plurality of second pixel values applied with the first weight value, filtering on the obtained image.

The one or more instructions, when executed by the at least one processor individually or collectively, cause the display apparatus to: obtain, based on the plurality of first pixel values corresponding to the facial area of the user and a plurality of third pixel values corresponding to a remaining area other than the facial area of the user identified from the obtained image, a contrast ratio between the facial area and the remaining area, apply a second weight value corresponding to the obtained contrast ratio to the plurality of second pixel values, and perform, based on a plurality of fourth pixel values corresponding to the obtained image and the plurality of second pixel values applied with the second weight value, filtering on the obtained image.

The one or more instructions, when executed by the at least one processor individually or collectively, cause the display apparatus to: identify a plurality of third pixel values corresponding respectively to a red channel, a blue channel, and a green channel of the obtained image, identify, based on hue values respectively set for a red color, a blue color, and a green color of the display apparatus, a plurality of third weight values corresponding respectively to a red channel, a blue channel, and a green channel of the content, apply the identified plurality of third weight values to the plurality of second pixel values, the plurality of second pixel values corresponding respectively to the red channel, the blue channel, and the green channel of the content, obtain a plurality of fifth pixel values corresponding respectively to the red channel, the blue channel, and the green channel of the corrected image by subtracting the plurality of second pixel values applied with the plurality of third weight values from a plurality of fourth pixel values, the plurality of fourth pixel values corresponding respectively to the red channel, the blue channel, and the green channel of the obtained image, and obtain the corrected image based on the plurality of fifth pixel values.

The one or more instructions, when executed by the at least one processor individually or collectively, cause the display apparatus to: obtain a plurality of images through the camera, based on the determination that filtering is to be performed on the obtained image, obtain a plurality of corrected images by performing filtering on the plurality of images obtained through the camera, obtain a moving average value of a signal-to-noise ratio of the biometric signal based on the obtained plurality of corrected images, and stop obtaining the plurality of images based on the moving average value being less than a pre-set third value for a pre-set time.

The one or more instructions, when executed by the at least one processor individually or collectively, cause the display apparatus to: identify, based on the plurality of first pixel values corresponding to the facial area of the user and a plurality of third pixel values corresponding to a remaining area other than the facial area of the user identified from the obtained image, a contrast ratio between the facial area and the remaining area, identify an average value of the plurality of second pixel values, the plurality of second pixel values corresponding to a green channel among a plurality of channels of the content, identify whether a correlation degree between the plurality of first pixel values and the plurality of second pixel values is greater than or equal to a pre-set first value, based on the correlation degree between the plurality of first pixel values and the plurality of second pixel values being greater than or equal to the pre-set first value, identify whether the contrast ratio is greater than or equal to a pre-set second value, based on the contrast ratio being greater than or equal to the pre-set second value, identify whether the average value of the plurality of second pixel values is greater than or equal to a pre-set third value, and based on the average value of the plurality of second pixel values being greater than or equal to the pre-set third value, determine to perform filtering on the obtained image.

According to an aspect of the disclosure, a method for controlling a display apparatus may be provided. The method includes, based on an occurrence of a pre-set event, obtaining an image through a camera of the display apparatus while content is being output through a display of the display apparatus; determining, based on a plurality of first pixel values corresponding to a facial area of a user identified in the obtained image and a plurality of second pixel values corresponding to the content displayed through the display, whether to perform filtering on the obtained image; based on determining that filtering is to be performed on the obtained image, obtaining a corrected image by performing filtering on the obtained image based on the plurality of second pixel values; and identifying a biometric signal of the user based on the corrected image.

The determining whether to perform filtering on the obtained image includes determining to perform filtering on the obtained image based on a correlation degree between the plurality of first pixel values and the plurality of second pixel values being greater than or equal to a pre-set first value.

The determining that filtering is to be performed on the obtained image includes identifying, based on the plurality of first pixel values corresponding to the facial area of the user and a plurality of third pixel values corresponding to a remaining area other than the facial area of the user identified from the obtained image, a contrast ratio between the facial area and the remaining area; and determining to perform filtering on the obtained image based on the identified contrast ratio being greater than or equal to a pre-set second value.

The determining whether to perform filtering on the obtained image includes determining to perform filtering on the obtained image based on an average value of the plurality of second pixel values being greater than or equal to a pre-set third value, the plurality of second pixel values corresponding to a green channel among a plurality of channels of the content.

The method further includes, for each image frame of a plurality of image frames of the content, identifying an average value of the plurality of second pixel values corresponding the a green channel of the image frame; identifying, based on each average value corresponding to each image frame, a frequency corresponding to a green channel of the content; and determining to perform filtering on the obtained image based on the identified frequency being within a pre-set range and each average value of each frame of the plurality of image frames of the content being greater than or equal to a pre-set third value.

The obtaining the corrected image includes applying a first weight value corresponding to a luminance setting value of the display apparatus to the plurality of second pixel values; and performing, based on a plurality of fourth pixel values corresponding to the obtained image and the plurality of second pixel values applied with the first weight value, filtering on the obtained image.

The obtaining the corrected image includes obtaining, based on the plurality of first pixel values corresponding to the facial area of the user and a plurality of third pixel values corresponding to a remaining area other than the facial area of the user identified from the obtained image, a contrast ratio between the facial area and the remaining area, applying a second weight value corresponding to the obtained contrast ratio to the plurality of second pixel values, and performing, based on a plurality of fourth pixel values corresponding to the obtained image and the plurality of second pixel values applied with the second weight value, filtering on the obtained image.

The obtaining the corrected image includes identifying a plurality of third pixel values corresponding respectively to a red channel, a blue channel, and a green channel of the obtained image, identifying, based on hue values respectively set for a red color, a blue color, and a green color of the display apparatus, a plurality of third weight values corresponding respectively to a red channel, a blue channel, and a green channel of the content, applying the identified plurality of third weight values to the plurality of second pixel values, the plurality of second pixel values corresponding respectively to the red channel, the blue channel, and the green channel of the content, obtaining a plurality of fifth pixel values corresponding respectively to the red channel, the blue channel, and the green channel of the corrected image by subtracting the plurality of second pixel values applied with the plurality of third weight values from a plurality of fourth pixel values, the plurality of fourth pixel values corresponding respectively to the red channel, the blue channel, and the green channel of the obtained image, and obtaining the corrected image based on the plurality of fifth pixel values.

The method further includes obtaining a plurality of images through the camera, based on determining that filtering is to be performed on the obtained image, obtaining a plurality of corrected images by performing filtering on the plurality of images obtained through the camera, obtaining a moving average value of a signal-to-noise ratio of the biometric signal based on the obtained plurality of corrected images, and stopping the obtaining the plurality of images based on the moving average value being less than a pre-set third value for a pre-set time.

According to an aspect of the disclosure, a non-transitory computer-readable medium storing instructions that, when executed by one or more processors of a display apparatus, cause the one or more processors to, based on an occurrence of a pre-set event, obtain an image through a camera of the display apparatus while content is being output through a display of the display apparatus, determine; based on a plurality of first pixel values corresponding to a facial area of a user identified in the obtained image and a plurality of second pixel values corresponding to the content, whether to perform filtering on the obtained image; based on a determination that filtering is to be performed on the obtained image, obtain a corrected image by performing filtering on the obtained image based on the plurality of second pixel values; and identify a biometric signal of the user based on the corrected image.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is an example diagram of a display apparatus according to one or more embodiments of the disclosure;

FIG. 2 is a schematic configuration diagram of a display apparatus according to one or more embodiments of the disclosure;

FIG. 3 is a flowchart schematically illustrating a control method of a display apparatus according to one or more embodiments of the disclosure;

FIG. 4 is a flowchart illustrating a method for controlling a display apparatus to determine whether to perform filtering based on a correlation degree between a facial area of a user and content according to one or more embodiments of the disclosure;

FIG. 5 is a flowchart illustrating a method for controlling a display apparatus to determine whether to perform filtering based on a contrast ratio of a remaining area in an obtained image and a facial area of a user according to one or more embodiments of the disclosure;

FIG. 6 is an example diagram illustrating an example of determining whether to perform filtering based on a contrast ratio of a remaining area in an obtained image and a facial area of a user according to one or more embodiments of the disclosure;

FIG. 7 is a flowchart illustrating a method for controlling a display apparatus to determine whether to perform filtering based on a plurality of second pixel values corresponding to a green channel of content according to one or more embodiments of the disclosure;

FIG. 8 is an example diagram illustrating controlling of a display apparatus to determine whether to perform filtering based on a plurality of second pixel values corresponding to a green channel of content according to one or more embodiments of the disclosure;

FIG. 9 is a flowchart illustrating a method for identifying whether to perform filtering according to one or more embodiments of the disclosure;

FIG. 10 is a flowchart illustrating a method for performing filtering based on a luminance setting value of a display apparatus according to one or more embodiments of the disclosure;

FIG. 11 is an example diagram illustrating a method for performing filtering based on setting information of a display apparatus according to one or more embodiments of the disclosure;

FIG. 12 is an example diagram illustrating performing filtering based on a weight value set based on a luminance setting value according to one or more embodiments of the disclosure;

FIG. 13 is an example diagram illustrating performing filtering based on a weight value set based on a hue value set to each red color, green color, and blue color according to one or more embodiments of the disclosure;

FIG. 14 is an example diagram illustrating adjusting of first and second weight values based on a contrast ratio according to one or more embodiments of the disclosure; and

FIG. 15 is a detailed block diagram of a display apparatus according to one or more embodiments of the disclosure.

### DETAILED DESCRIPTION

Various modifications may be made to the embodiments of the disclosure, and there may be various types of embodiments. Accordingly, specific embodiments will be illustrated in drawings, and described in detail in the detailed description. However, it should be noted that the various embodiments are not for limiting the scope of the disclosure to a specific embodiment, but they should be understood to include various modifications, equivalents or alternatives of the embodiments of the disclosure. With respect to the description of the drawings, like reference numerals may be used to indicate like elements.

In describing the disclosure, in case it is determined that the detailed description of related known technologies or configurations may unnecessarily confuse the gist of the disclosure, the detailed description thereof will be omitted.

Further, the embodiments below may be modified to various different forms, and it is to be understood that the scope of the technical spirit of the disclosure is not limited to the embodiments below. Rather, the embodiments are provided so that the disclosure will be thorough and complete, and to fully convey the technical spirit of the disclosure to those skilled in the art.

Terms used in the disclosure have been merely used to describe a specific embodiment, and is not intended to limit the scope of protection. A singular expression includes a plural expression, unless otherwise specified.

In the disclosure, expressions such as “have”, “may have”, “include”, and “may include” are used to designate a presence of a corresponding characteristic (e.g., elements such as numerical value, function, operation, or component), and not to preclude a presence or a possibility of additional characteristics.

In the disclosure, expressions such as “A or B”, “at least one of A and/or B”, or “one or more of A and/or B” may include all possible combinations of the items listed together. For example, “A or B”, “at least one of A and B”, or “at least one of A or B” may refer to all cases including (1) at least one A, (2) at least one B, or (3) both of at least one A and at least one B.

As used herein, the terms “1st” or “first” and “2nd” or “second” may use corresponding components regardless of importance or order and are used to distinguish one component from another without limiting the components.

When a certain element (e.g., first element) is indicated as being “(operatively or communicatively) coupled with/to” or “connected to” another element (e.g., second element), it may be understood as the certain element being directly coupled with/to the another element or as being coupled through other element (e.g., third element).

Conversely, when the certain element (e.g., first element) is indicated as “directly coupled with/to” or “directly connected to” another element (e.g., second element), it may be understood as the other element (e.g., third element) not being present between the certain element and the another element.

The expression “configured to . . . (or set up to)” used in the disclosure may be used interchangeably with, for example, “suitable for . . . ”, “having the capacity to . . . ”, “designed to . . . ”, “adapted to . . . ”, “made to . . . ”, or “capable of . . . ” based on circumstance. The term “configured to . . . (or set up to)” may not necessarily mean “specifically designed to” in terms of hardware.

Rather, in a certain circumstance, the expression “an apparatus configured to . . . ” may mean something that the apparatus “may perform . . . ” together with another apparatus or components. For example, a phrase “a sub-processor configured to (or set up to) perform A, B, or C” may mean a dedicated processor (e.g., an embedded processor) for performing a relevant operation, or a generic-purpose processor (e.g., a central processing unit (CPU) or an application processor) capable of performing the relevant operations by executing one or more software programs stored in a memory 160 apparatus.

The term ‘module’ or ‘part’ used in the embodiments perform at least one function or operation, and may be implemented with hardware or software, or implemented with a combination of hardware and software. In addition, a plurality of ‘modules’ or a plurality of ‘parts’, except for a ‘module’ or a ‘part’ which needs to be implemented with a specific hardware, may be integrated in at least one module and implemented as at least one processor.

Meanwhile, the various elements and areas in the drawings have been schematically illustrated. Accordingly, the technical spirit of the disclosure is not limited by relative sizes and distances illustrated in the accompanied drawings.

An embodiment of the disclosure will be described in detail below with reference to the accompanied drawings to aid in the understanding of those of ordinary skill in the art.

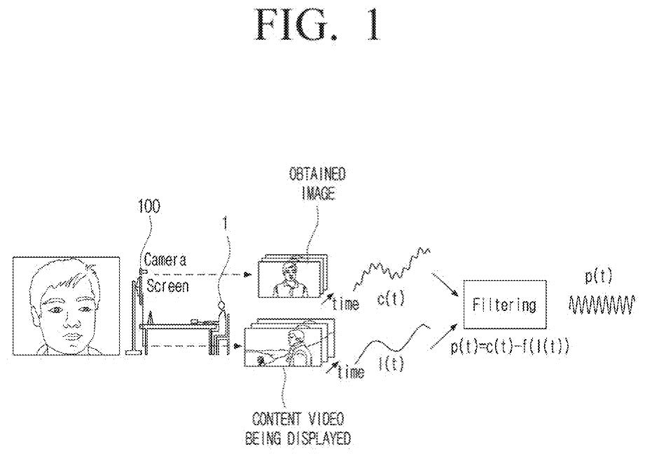

FIG. 1 is an example diagram of a display apparatus 100 according to an embodiment of the disclosure.

Referring to FIG. 1, due to recent developments in electronic technology, the display apparatus 100 has been able to exceed from its function of displaying only existing videos (e.g., content), and perform even a home care function of obtaining biometric information about a user 1 and analyzing a health state of the user 1 based on the obtained biometric information. Specifically, the display apparatus 100 may obtain an image of the user 1 through a camera provided at one surface of the display apparatus 100 and then, obtain biometric information of the user 1 by analyzing the obtained image. For example, the display apparatus 100 may analyze a plurality of images obtained according to time, identify changes in skin color of the user 1, and obtain biometric information such as a blood flow rate, a pulse rate, blood pressure, and the like of the user 1 based on the identified changes in skin color.

Accordingly, it is important to obtain an accurate image of the user 1. However, if an image of the user 1 is obtained through the camera while the display apparatus 100 is displaying a video, information (e.g., skin color) about the user 1 included in the obtained image may be distorted. In other words, an inaccurate image of the user may be obtained.

Specifically, while the display apparatus 100 is displaying a video, light by the video output from the display apparatus 100 may be emitted. At this time, when the emitted light reaches a subject of the camera, noise information by the emitted light may be included in obtained image. Specifically, when the emitted light reaches the user 1 using the display apparatus 100, information about the user 1 included in the obtained image may be distorted information affected by the emitted light. Accordingly, with respect to the image of the user 1 obtained while the display apparatus 100 is displaying the video, there is a need for a method to remove noise information included in the image or correct the distorted information about the user.

To solve the above-described problem, the display apparatus 100 according to an embodiment of the disclosure may analyze the video displayed in the display apparatus 100, and identify whether light emitted from the display apparatus 100 according to displaying the video affects the obtained image of the user 1. Further, the display apparatus 100 may perform, based on light emitted from the display apparatus 100 being identified as affecting the obtained image of the user 1, a filtering process on the image of the user 1. The filtering process may include removing noise components by the emitted light in the obtained image of the user 1.

Specifically, because light emitted from the display apparatus 100 is by the video displayed in the display apparatus 100, the display apparatus 100 may perform the filtering process based on the displayed video. In an example, referring to FIG. 1, the display apparatus 100 may perform the filtering process on the obtained image of the user by removing a noise signal f(l(t)) according to a video signal l(t) of the video displayed in the display apparatus 100 from a video signal c(t) of a plurality of obtained user images. At this time, the display apparatus 100 may identify the noise signal f(l(t)) through applying weight values to the video signal l(t) of the video displayed in the display apparatus 100 or through normalization, vectorization, and the like of the video signal l(t). Meanwhile, the display apparatus 100 may accurately obtain biometric information of the user through a video signal p(t) of a corrected image obtained through the filtering process.

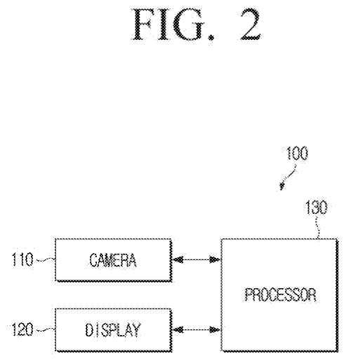

One or more embodiments of the disclosure will be described in detail below with reference to FIG. 2 to FIG. 14.

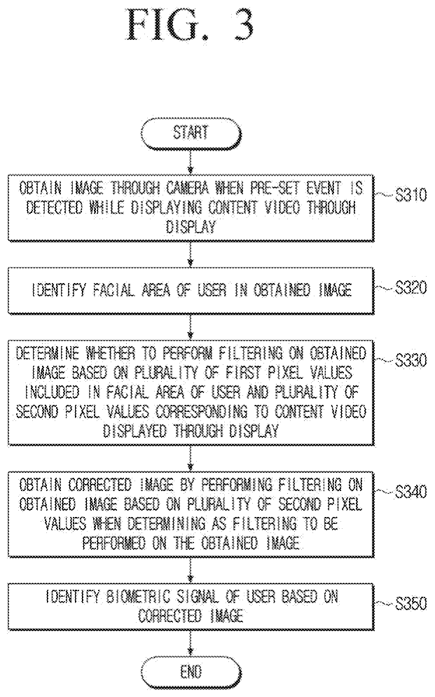

FIG. 2 is a schematic configuration diagram of the display apparatus 100 according to an embodiment of the disclosure. FIG. 3 is a flowchart schematically illustrating a control method of the display apparatus 100 according to an embodiment of the disclosure.

Referring to FIG. 2, the display apparatus 100 according to an embodiment of the disclosure may include a camera 110, a display 120, and a processor 130.

The display apparatus 100 according to an embodiment of the disclosure may be an apparatus for displaying content composed of a plurality of image frames, and for example, the display apparatus 100 may be implemented as various display apparatuses 100 such as, for example, and without limitation, a TV, a smart TV, signage, a desktop PC, a notebook, a smart phone, a tablet PC, and the like.

The camera 110 may obtain an image about an object by capturing the object at the surrounding of the display apparatus 100. Specifically, the camera 110 may obtain a plurality of images about a user positioned at the surrounding of the display apparatus 100. To this end, the camera 110 may be implemented as a capturing device having a CMOS sensor (CMOS image sensor (CIS)), a capturing device having a CCD structure (charge coupled device), and the like. However, the embodiment is not limited thereto, and the camera 110 may be implemented as a camera 110 module of various resolutions capable of capturing a subject.

Meanwhile, the camera 110 may be implemented as a depth camera 110 (e.g., IR depth camera 110, etc.), a stereo camera 110, an RGB camera 110, or the like. Thereby, in an image obtained through the camera 110, depth information about an object (e.g., user) may be further included.

The display 120 may display various visual information (e.g., content) according to control by the processor 130. Here, the content may include videos of various formats such as, for example, and without limitation, a text, a still video, a moving video, a graphic user interface (GUI), and the like. The display 120 may be implemented with a touch screen together with a touch panel. At this time, the display 120 may function as an output part outputting information between the display apparatus 100 and the user while simultaneously functioning as an input part providing an input interface between the display apparatus 100 and the user.

The display 120 may be implemented as displays 120 of various forms such as, for example, and without limitation, a liquid crystal display (LCD) panel, light emitting diode (LED), Organic Light Emitting Diodes (OLED), Liquid Crystal on Silicon (LCOS), Digital Light Processing (DLP), and the like. The display 120 may additionally include additional configurations according to an implementation method thereof. For example, the display 120 may be further included with a driving circuit that is implementable in a form such as an a-si TFT, a low temperature poly silicon (LTPS) TFT, an organic TFT (OTFT), and the like, and a back light unit, and the like.

The processor may control an overall operation and function of the display apparatus 100 by being electrically connected with the camera 110 and the display 120.

The processor 130 may include one or more among a central processing unit (CPU), a graphics processing unit (GPU), an accelerated processing unit (APU), a many integrated core (MIC), a digital signal processor (DSP), a neural processing unit (NPU), a hardware accelerator, or a machine learning accelerator. The processor 130 may control one or a random combination among other elements of the display apparatus 100, and perform an operation associated with communication or data processing. The processor 130 may execute one or more programs or instructions stored in memory (not shown). For example, the processor 130 may perform, by executing one or more instructions stored in the memory, a method according to an embodiment of the disclosure.

When a method according to an embodiment of the disclosure includes a plurality of operations, the plurality of operations may be performed by one processor, or performed by a plurality of processors. For example, when a first operation, a second operation, and a third operation are performed by a method according to an embodiment, the first operation, the second operation, and the third operation may all be performed by a first processor, or the first operation and the second operation may be performed by the first processor (e.g., generic-purpose processor) and the third operation may be performed by a second processor (e.g., artificial intelligence dedicated processor).

The processor 130 may be implemented as a single core processor that includes one core, or implemented as one or more multicore processors that include a plurality of cores (e.g., homogeneous multicore or heterogeneous multicore). If the processor 130 is implemented as multicore processors, each of the plurality of cores included in the multicore processors may include a memory inside the processor 130 such as a cache memory and an on-chip memory, and a common cache shared by the plurality of cores may be included in the multicore processors. In addition, each of the plurality of cores (or a portion among the plurality of cores) included in the multicore processors may independently read and perform a program command for implementing a method according to an embodiment of the disclosure, or read and perform a program command for implementing a method according to an embodiment of the disclosure due to a whole (or a portion) of the plurality of cores being interconnected.

When a method according to an embodiment of the disclosure includes a plurality of operations, the plurality of operations may be performed by one core among the plurality of cores or performed by the plurality of cores included in the multicore processors. For example, when a first operation, a second operation, and a third operation are performed by a method according to an embodiment, the first operation, the second operation, and the third operation may all be performed by a first core included in the multicore processors, or the first operation and the second operation may be performed by the first core included in the multicore processors and the third operation may be performed by a second core included in the multicore processors.

In an embodiment of the disclosure, the processor 130 may refer to a system on chip (SoC), a single core processor, or multicore processors in which one or more processors and other electronic components are integrated or a core included in the single core processor or the multicore processors, and the core herein may be implemented as the CPU, the GPU, the APU, the MIC, the DSP, the NPU, the hardware accelerator, the machine learning accelerator, or the like, but the embodiments of the disclosure are not limited thereto.

The processor 130 may obtain, when a pre-set event occurs while outputting content through the display 120, an image through the camera 110 (S310).

Specifically, the processor 130 may display content through the display 120. The content may be a video received from a source apparatus or a server apparatus connected with the display apparatus 100. For example, the display apparatus 100 may receive the content from the source apparatus that is connected via an HDMI cable. Alternatively, the display apparatus 100 may receive the content from the server apparatus through a communication interface. The server apparatus that provides the content may be an OTT platform server apparatus.

The processor 130 may display, when receiving a user command requesting to display content through the input interface of the display apparatus 100 or a control command (e.g., control command requesting to display content) from an external control apparatus that operates in connection with the display apparatus 100 through the communication interface, content through the display 120. Alternatively, the processor 130 may also display content through the display 120 according to information (e.g., reservation setting for content) set in the display apparatus 100.

At this time, the processor 130 may detect that a pre-set event has occurred while displaying content through the display 120. The pre-set event may be an event set for the processor 130 to obtain an image of the user positioned at a front direction of the display apparatus 100 through the camera 110. The pre-set event may include, as content is displayed in the display 120, an event in which a color of a light source emitted from the display 120 is determined as changed. For example, the pre-set event may include a change in channel or content of the display apparatus 100, a change in program (or application) executed in the display apparatus 100, and the like.

The processor 130 may obtain, when detecting the occurrence of the pre-set event, an image through the camera 110. Specifically, the processor 130 may capture the user using the display apparatus 100 through the camera 110, and obtain a plurality of images of the user. To this end, the camera 110 may be provided at one surface of the front surface of the display apparatus 100, and the processor 130 may capture the user positioned at the front direction of the display apparatus 100 through the camera 110, and obtain a plurality of images of the user.

Specifically, the processor 130 may obtain a plurality of images of a face of the user. To this end, the processor may track the face of the user through the camera 110, and obtain the plurality of images of the face of the user. For example, the processor 130 may recognize the face of the user included in the plurality of images by applying a motion vector technique, an optical flow technique, a particle filter technique, and the like to the plurality of images obtained through the camera 110, perform continuous tracking of the face of the user, and also obtain a plurality of images of the face of the user.

Meanwhile, the processor 130 may identify whether the pre-set event has been detected for every pre-set time (periodically). At this time, the processor 130 may obtain an image of the user through the camera 110 even if the pre-set event has not been detected for a pre-set time, and the pre-set event has not occurred.

Then, the processor may determine, based on a plurality of pixel values included in an identified facial area of the user in the obtained image and a plurality of pixel values corresponding to the content, whether to perform filtering on the obtained image (S320).

Specifically, the processor 130 may identify the facial area of the user in the obtained image. The processor 130 may set the face of the user in the obtained image as an area of interest. For example, the processor 130 may detect the facial area of the user in the image as an area of interest through template matching, or detect a boundary line of the face of the user through contour detection, and detect the facial area of the user as the area of interest. In addition thereto, the processor 130 may detect the face of the user in the image through a deep learning model (e.g., You Only Look Once) model, a faster R-CNN model, and a single shot multi box detector (SSD)), a region-based segmentation algorithm, and identify the area with respect to the face of the user as the area of interest.

Then, the processor 130 may determine, based on a plurality of pixel values included in the facial area of the user and a plurality of pixel values corresponding to content displayed through the display 120, whether to perform filtering on the obtained image.

Specifically, the processor 130 may identify each pixel value of a plurality of pixels included in the facial area of the user in the image. The pixel value may be a value that quantified information about color, brightness, and the like that each pixel has. The pixel value may include a luminance value of a pixel, a grayscale value of a pixel, color coordinate values of a pixel, and the like.

If the image obtained through the camera 110 is a color image, the processor 130 may identify a pixel value of an image for three channels that correspond respectively to a red color, a green color, and a blue color. Specifically, the processor 130 may identify a pixel value of the red color (i.e., pixel value corresponding to a red channel), identify a pixel value of the green color (i.e., pixel value corresponding to a green channel), and identify a pixel value of the blue color (i.e., pixel value corresponding to blue channel). For convenience of description below, pixels included in the facial area of the user identified in the image may be referred to as a first pixel, and a pixel value of the first pixel as a first pixel value.

In addition, the processor 130 may identify a plurality of pixel values corresponding to content that is displayed through the display 120. Here, the plurality of pixel values corresponding to content may be respective pixel values that the plurality of pixels have included in the display 120 based on displaying the content. The plurality of pixel values corresponding to content may be different according to the plurality of image frames that constitute the content. The processor 130 may receive pixel value information of the content together with the content from the source apparatus or the server apparatus that provides the content. Meanwhile, because the description for the plurality of pixel values included in the facial area of the user in the above-described image may be identically applied to the plurality of pixel values corresponding to content, detailed description thereof will be omitted.

For convenience of description below, pixels corresponding to content may be referred to as second pixels, and a pixel value of the second pixel may be referred to as a second pixel value.

The processor 130 may determine, based on a plurality of first pixel values included in the facial area of the user in the image and a plurality of second pixel values corresponding to content, whether to perform filtering process on the obtained image through the camera 110.

Specifically, the processor 130 may determine, while displaying content through the display 120, whether light emitted from the display 120 is identified as affecting the image obtained through the camera 110. The processor 130 may determine whether light emitted from the display 120 is identified as affecting the image obtained through the camera 110 based on the plurality of first pixel values included in the facial area in the image and the plurality of second pixel values. Specifically, as content is displayed through the display 120, light is emitted from the display 120, and if the emitted light reaches the face of the user using the display apparatus 100, distorted information about the face of the user may be included in the plurality of first pixel values of the obtained image.

Accordingly, the processor 130 may determine, based on the plurality of first pixel values included in the facial area of the user in the image and the plurality of second pixel values corresponding to the content, whether light emitted from the display 120 is identified as affecting the image obtained through the camera 110, and if the light emitted from the display 120 is identified as affecting the image obtained through the camera 110, determine that the filtering process is to be performed on the image obtained through the camera 110.

At this time, the processor 130 may identify an image frame that matches with an image obtained through the camera 110 among a plurality of image frames that include the content that is displayed through the display 120. Specifically, the processor 130 may extract a plurality of image frames of a content displayed through the display 120 within a pre-set time range from a time-point at which the image is obtained through the camera 110. Then, the processor 130 may identify each correlation degree between the obtained image and the plurality of image frames.

In an example, the processor 130 may identify a correlation degree by calculating a cross-correlation value between the obtained image and the plurality of image frames. Specifically, the processor 130 may obtain a summed value after multiplying a pixel value of the obtained image with the second pixel value of the respective image frames after moving (or sliding) the obtained image on the plurality of image frames. Then, the processor 130 may identify the summed value obtained for each image frame as a correlation degree between each image frame and the obtained image. Then, the processor 130 may identify the image frame with the highest correlation degree, and identify the identified image frame with the highest correlation degree as an image frame that matches with the obtained image. Specifically, the processor 130 may synchronize or calibrate a time-point at which the image frame with the highest correlation degree is displayed and the time-point at which the image is obtained, and based on a filtering process between a plurality of image frames of the content that is displayed thereafter and a plurality of images, determine whether to perform the filtering process.

An embodiment of the disclosure on determining whether to perform the filtering process will be described below.

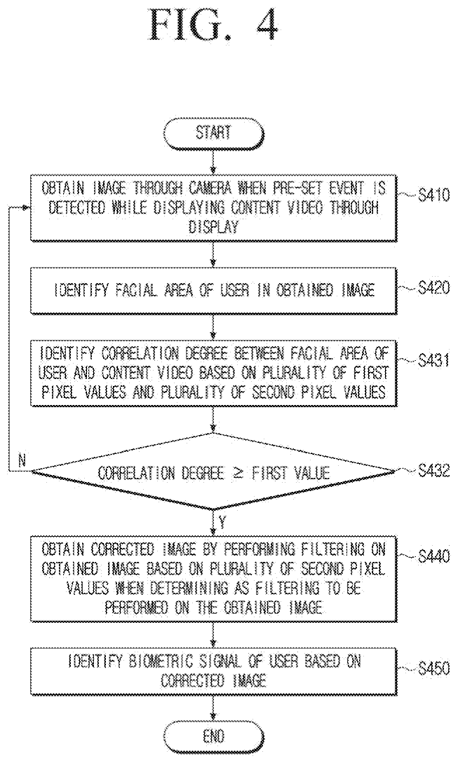

FIG. 4 is a flowchart illustrating a method for controlling the display apparatus 100 to determine whether to perform filtering based on a correlation degree between a facial area of a user and content according to an embodiment of the disclosure. Steps S410, S440, and S450 shown in FIG. 4 may correspond respectively to steps S310, S330, and S340 shown in FIG. 3.

According to an embodiment of the disclosure, the processor 130 may identify, based on the plurality of first pixel values and the plurality of second pixel values, a correlation degree between the facial area of the user and the content (S431), and determine to perform filtering on the obtained image based on the correlation degree being greater than or equal to a pre-set first value (S432).

Specifically, the processor may identify a correlation degree between the facial area in the obtained image and the content based on the plurality of first pixel values included in the facial area in the obtained image and the plurality of second pixel values corresponding to the content. Specifically, the processor 130 may identify the correlation degree by calculating the cross-correlation value between the image corresponding to the facial area in the obtained image and the image frame included in the content. At this time, the image frame that identifies the correlation degree with the image corresponding to the facial area may be an image frame that matches with an image obtained among a plurality of image frames that constitute the above-described content.

The processor 130 may obtain, by moving (sliding) the image corresponding to the facial area on the image frame of the content, the summed value after multiplying the plurality of first pixel values included in the facial area and the plurality of second pixel values corresponding to the content. Then, the processor 130 may identify the obtained summed value as the correlation degree between the facial area in the obtained image and the content.

At this time, the processor 130 may identify, based on the pixel value of the image for the three channels that correspond respectively to the red color, the green color, and the blue color, the correlation degree of the image frame included in the content for each channel. The processor 130 may identify a plurality of third pixel values included in the facial area of the image obtained through the camera 110 according to a plurality of channels (i.e., red channel, green channel, and blue channel). Specifically, the processor 130 may separate the obtained image according to the red channel, the green channel, and the blue channel, and identify the plurality of first pixel values for each separated image (i.e., each of the images corresponding to the red channel, the green channel, and the blue channel). In other words, the processor 130 may identify the plurality of first pixel values corresponding to the red channel, identify the plurality of first pixel values corresponding to the green channel of the obtained image, and identify the plurality of first pixel values corresponding to the blue channel of the obtained image.

Then, the processor 130 may identify, for even the image frame of the content, the second pixel value of the image for the three channels that correspond respectively to the red color, the green color, and the blue color. In other words, the processor 130 may identify the plurality of second pixel values included in the image frame that matches with the image obtained through the camera 110 according to the plurality of channels (i.e., red channel, green channel, and blue channel). Specifically, the processor 130 may separate the image frame of the content according to the red channel, the green channel, and the blue channel, and identify the plurality of second pixel values for each separated image frame (i.e., each image corresponding to the red channel, the green channel, and the blue channel). The processor 130 may identify the plurality of second pixel values corresponding to the red channel of the image frame in the content, identify the plurality of second pixel values corresponding to the green channel of the obtained image, and identify the plurality of second pixel values corresponding to the blue channel of the obtained image.

Then, the processor 130 may obtain each of a correlation degree of the red channel, a correlation degree of the green channel, and a correlation degree of the blue channel. Specifically, the processor 130 may obtain the correlation degree of the red channel based on the plurality of first pixel values corresponding to the red channel of the obtained image and the plurality of second pixel values corresponding to the red channel of the image frame of the content. Specifically, the processor 130 may move (slide) the image corresponding to the red channel obtained by separating the obtained image on the image corresponding to the red channel obtained by separating the image frame of the content, obtain the summed value after multiplying the plurality of first pixel values with the plurality of second pixel values corresponding to the red channel of the image frame, and identify the obtained value as the correlation degree of the red channel (first correlation degree). Likewise, the processor 130 may respectively identify the correlation degree of the green channel (second correlation degree) and the correlation degree of the blue channel (third correlation degree).

The processor 130 may determine that filtering is to be performed on the obtained image based on the correlation degree being greater than or equal to a pre-set first value. In other words, the processor 120 may determine as performing filtering on the obtained image based on the summed value after multiplying the plurality of second pixel values corresponding to the content with the plurality of first pixel values included in the facial area being greater than or equal to the pre-set first value.

Meanwhile, if the processor 130 determines whether to perform filtering based on a relevance degree of the plurality of channels (first relevance degree of the red channel, second relevance degree of the green channel, and third relevance degree of the blue channel), the processor 130 may respectively identify whether a plurality of relevance degrees is greater than or equal to the pre-set first value. At this time, the pre-set first value for the plurality of channels may be set as different values from one another. Specifically, the processor 130 may adjust a first value set for each channel based on an average second pixel value of each channel of the content.

Meanwhile, the first value set with respect to the correlation degree may be set based on color, size and the like of the face of the user. Specifically, the processor 130 may identify, every time an image is obtained through the camera 110, the skin color, the size and the like of the face of the user by analyzing the face of the user identified in the image, and set the first value based on the identified color, size, and the like of the face of the user. Specifically, the processor 130 may identify features (i.e., skin color, size, etc.) of the face of the user for each user if the user that uses the display apparatus 100 is in plurality, and set different values according to the features of the face of the user.

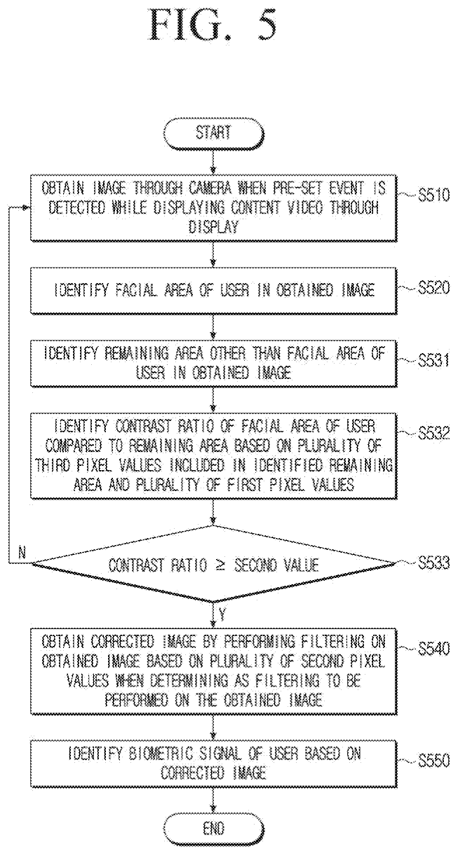

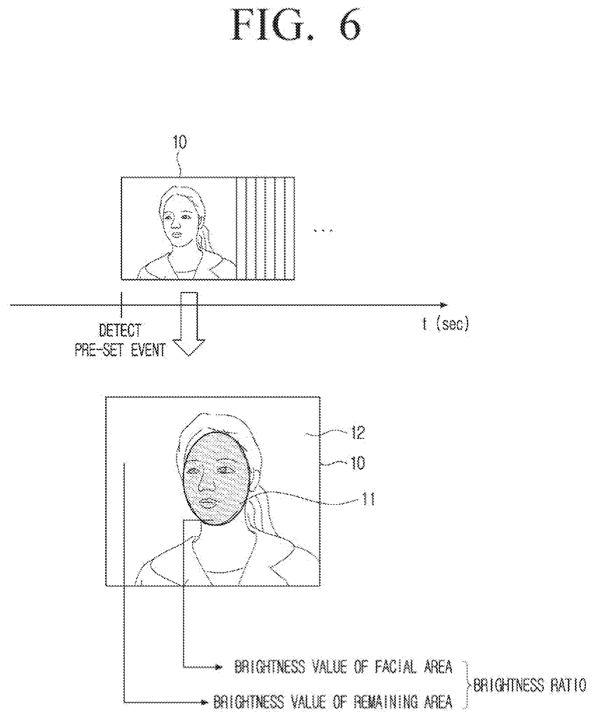

FIG. 5 is a flowchart illustrating a method for controlling the display apparatus 100 to determine whether to perform filtering based on a contrast ratio of a remaining area in an obtained image and a facial area of a user according to an embodiment of the disclosure. FIG. 6 is an example diagram illustrating an example of determining whether to perform filtering based on a contrast ratio of a remaining area in an obtained image and a facial area of a user according to an embodiment of the disclosure.

According to an embodiment of the disclosure, the processor 130 may identify the remaining area other than the facial area of the user in the obtained image (S531), identify a contrast ratio of the facial area of the user compared to the remaining area based on the plurality of third pixel values included in the identified remaining area and the plurality of first pixel values (S532), and determine that filtering is to be performed on the obtained image based on the identified contrast ratio being greater than or equal to a pre-set second value (S533).

Specifically, referring to FIG. 6, the processor 130 may identify a remaining area 12 other than a facial area 11 of a user identified in the obtained image 10. Here, the remaining area 12 may be an area other than the facial area 11 of the user set as the area of interest. For example, the remaining area 12 may include the background, remaining body parts other than the face of the user, and the like. Then, the processor 130 may identify a pixel value of the plurality of pixels included in the remaining area 12. For convenience of description below, the pixels included in the remaining area 12 may be referred to as a third pixel, and a pixel value of the third pixel may be referred to as a third pixel value.

The processor 130 may calculate the contrast ratio of the facial area 11 compared to the remaining area 12 based on the plurality of third pixel values included in the remaining area 12 and the plurality of first pixel values included in the facial area 11. In an example, the processor 130 may respectively calculate an average value of the plurality of first pixel values included in the facial area 11 and an average value of the plurality of third pixel values included in the remaining area 12, and obtain the calculated average value of the plurality of first pixel values compared to the calculated average value of the plurality of third pixel values as the contrast ratio of the facial area 11 compared to the remaining area 12. Meanwhile, as an average value of the pixel value is higher, brightness of a relevant area may be identified as low.

At this time, the processor 130 may calculate the contrast ratio of the facial area 11 compared to the remaining area 12 based on the plurality of pixel values corresponding to the plurality of channels (i.e., the plurality of first pixel values included in the facial area 11 (specifically, the plurality of first pixel values of the red channel, the plurality of first pixel values of the green channel, and the plurality of first pixel values of the blue channel) and the plurality of third pixel values included in the remaining area 12 (specifically, the plurality of third pixel values of the red channel, the plurality of third pixel values of the green channel, and the plurality of third pixel values of the blue channel)). In an example, the processor 130 may respectively calculate the average value of the plurality of first pixel values of the red channel, the plurality of first pixel values of the green channel, and the plurality of first pixel values of the blue channel for the facial area 11, and obtain a luminance value of the facial area 11 by re-calculating an average value of the calculated plurality of average values. Likewise, the processor 130 may obtain a brightness ratio of the facial area 11 compared to the remaining area 12 after also obtaining a luminance value of the remaining area 12.

Meanwhile, the processor 130 may determine that filtering is to be performed on the obtained image 10 based on the identified contrast ratio of the facial area 11 compared to the remaining area 12 being greater than or equal to the pre-set second value. The processor 130 may identify, based on the contrast ratio of the facial area 11 compared to the remaining area 12 being small, a difference in brightness of the facial area 11 of the user being great compared to the remaining area 12 in the image 10. Specifically, if the facial area 11 of the user is darker than the remaining area 12 (e.g., background, etc.), because the area may be more greatly affected by the light emitted from the display 120, the processor 130 may determine whether to perform filtering on the obtained image 10 based on the contrast ratio. At this time, the pre-set second value may be set differently according to the time, season, and the like at which the image 10 was obtained.

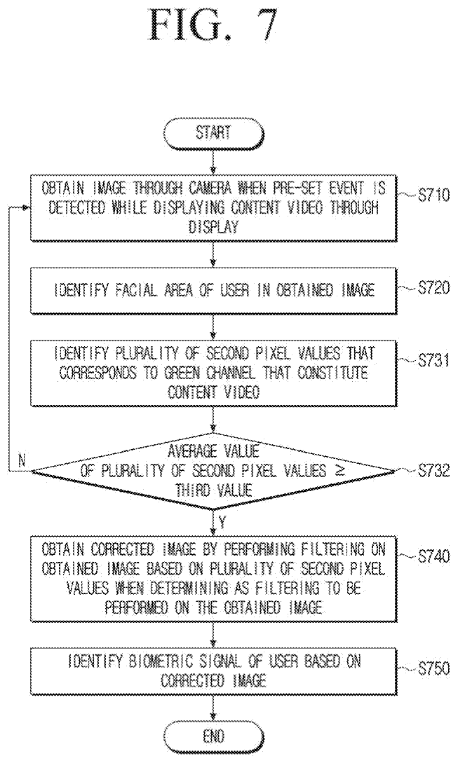

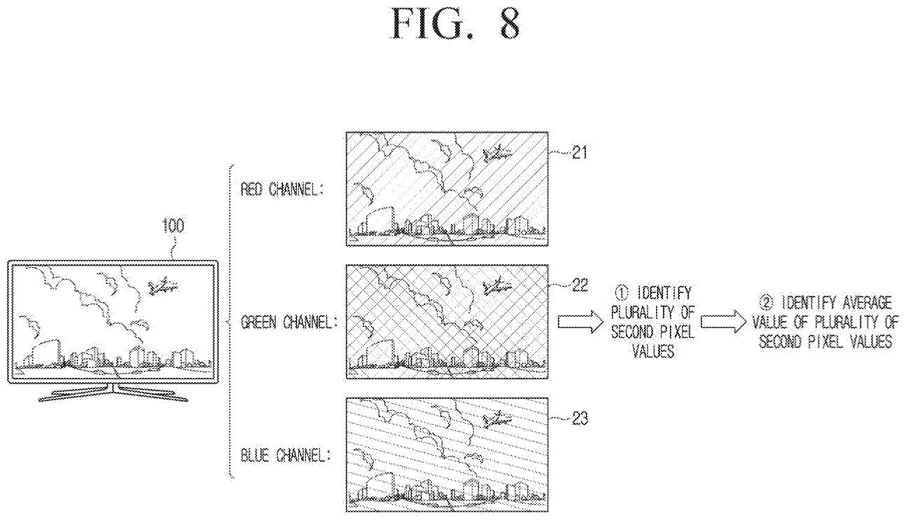

FIG. 7 is a flowchart illustrating a method for controlling the display apparatus 100 to determine whether to perform filtering based on a plurality of second pixel values corresponding to a green channel of content according to an embodiment of the disclosure. FIG. 8 is an example diagram illustrating controlling of the display apparatus 100 to determine whether to perform filtering based on a plurality of second pixel values corresponding to a green channel of content according to an embodiment of the disclosure.

Meanwhile, the processor 1030 may determine whether to perform filtering based on characteristics of the green channel among the plurality of channels about the content. Hemoglobin in blood of the user may react sensitively to, specifically, light of a green wavelength. Accordingly, light of the green wavelength emitted from the display 120 that displays content may also affect the hemoglobin in the blood of the face of the user. Ultimately, while light of the green wavelength is being emitted from the display 120, accurate information (e.g., movement of blood vessels in the face, pulse wave signals, etc.) about the face of the user may not be included in the image 10 obtained through the camera 110. Accordingly, the processor 130 may determine whether to perform the filtering process by identifying the characteristics of the green channel of the content that emits light of the green wavelength from the display 120. An embodiment of the disclosure associated therewith will be described in detail below.

The processor 130 according to an embodiment of the disclosure may identify the plurality of second pixel values corresponding to the green channel among the plurality of channels of the content (S731), and determine that filtering is to be performed on the obtained image 10 based on the identified average value of the plurality of second pixel values being identified as greater than or equal to a pre-set third value (S732).

Specifically, referring to FIG. 8, the processor 130 may identify the plurality of second pixel values corresponding to the green channel among the plurality of channels (i.e., red channel, green channel, and blue channel) of the content. As described above, the processor 130 may separate the content being displayed through the display 120 into the plurality of channels (i.e., red channel, green channel, and blue channel), and identify a pixel value of a plurality of second pixels (i.e., second pixel value) included in an image frame 22 about the green channel after obtaining image frame 22 about the green channel. Then, the processor 130 may calculate an average value of the identified plurality of second pixel values. At this time, the processor 130 may determine that filtering is to be performed on the obtained image 10 based on the calculated average value (i.e., average value of the plurality of second pixel values included in the image frame 22 about the green channel) being identified as greater than or equal to the pre-set third value. In other words, the processor 130 may identify that intensity of light of the green wavelength emitted from the display 120 is great based on the average value of the plurality of second pixel values included in the image frame 22 about the green channel being greater than or equal to the pre-set third value, and determine that filtering is to be performed on the obtained image 10.

In addition, according to an embodiment of the disclosure, the average value of the plurality of second pixel values corresponding to the green channel of the plurality of images of the content may be respectively identified, and frequency corresponding to the green channel of the content may be identified based on the plurality of average values corresponding to each of the image frames.

Specifically, the processor 130 may identify, based on the pre-set event being detected (or based on the average value of the plurality of second pixel values corresponding to the green channel being identified as greater than or equal to the pre-set third value), the average value of the plurality of second pixel values included in the green channel of the plurality of image frames included in the content that is displayed through the display 120 for a pre-set time. The processor 130 may identify the pixel value of the plurality of second pixels (i.e., second pixel value) included in the green channel of each of the image frames, and identify the average value of the plurality of second pixel values corresponding to the green channel of each of the image frames. Then, based on the identified average value of the plurality of second pixel values, the processor 130 may identify a video signal about the green channel of the content. Specifically, the processor 130 may identify a video signal g (t) about the green channel of the content according to time based on a plurality of image frames and an average value (specifically, the average value of the plurality of second pixel values) of each of the plurality of image frames.

Then, the processor 130 may identify, based on the identified video signal g (t), a frequency corresponding to the green channel of the content. Specifically, the processor 130 may identify a frequency of the identified video signal about the green channel. To this end, the processor 130 may detect at least one frequency component included in the identified video signal about the green channel. For example, the processor 130 may detect the at least one frequency component included in the video signal through a Fast Fourier Transform (FFT) of the identified video signal.

At this time, the processor 130 may determine that filtering is to be performed on the obtained image 10 based on the frequency corresponding to the green channel of the content being identified as within a pre-set range. Specifically, the processor 130 may determine that filtering is to be performed on the obtained image 10 based on the average value of the plurality of second pixel values corresponding to the green channel being greater than or equal to the pre-set third value, and the frequency corresponding to the green channel of the content being identified as within the pre-set range.

The processor 130 may identify whether a frequency of the identified video signal about the green channel is similar with a frequency of a pulse wave signal. To this end, the processor 130 may identify whether the frequency of the identified video signal about the green channel is within the pre-set range. The pre-set range may be set based on a pulse wave signal. For example, the pre-set range may be set from 0.5 Hz to 3.5 Hz. The processor 130 may identify that the frequency of the identified video signal about the green channel is similar with the frequency of the pulse wave signal based on the frequency of the identified video signal about the green channel being identified as greater than or equal to 0.5 Hz and less than 3.5 Hz, and determine that filtering is to be performed on the obtained image 10.

More specifically, the processor 130 may detect at least one frequency component included in the video signal through the Fast Fourier Transform (FFT) of the identified video signal, and determine that filtering is to be performed on the obtained image 10 based on any one among the detected at least one frequency component being identified as greater than or equal to 0.5 Hz and less than 3.5 Hz. Meanwhile, as described above, the processor 130 may determine whether to perform filtering by identifying whether the frequency corresponding to the green channel of the content is within the pre-set range based on the average value of the plurality of second pixel values corresponding to the green channel being identified as greater than or equal to the pre-set third value.

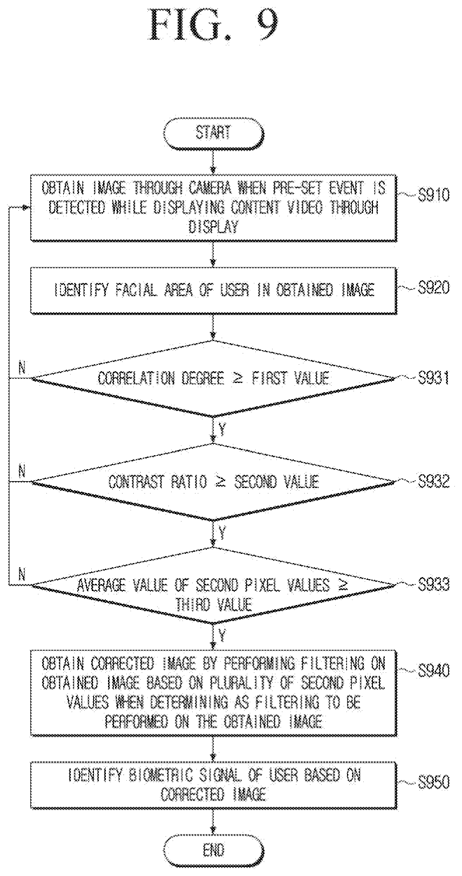

FIG. 9 is a flowchart illustrating a method for identifying whether to perform filtering according to an embodiment of the disclosure.

Meanwhile, according to an embodiment of the disclosure, the processor 130 may determine as performing the filtering process on the obtained image 10 based on conditions for determining whether to perform the above-described filtering all being satisfied. Specifically, the processor 130 may determine as performing filtering on the obtained image 10 based on the correlation degree between the facial area 11 of the user and the content being greater than or equal to the pre-set first value, a contrast ratio of the facial area 11 of the user compared to the remaining area 12 in the obtained image 10 being greater than or equal to the pre-set second value, the average value of the plurality of second pixel values corresponding to the green channel of the content being greater than or equal to the pre-set third value, and the frequency corresponding to the green channel of the content being identified as within the pre-set range. Because the above-described descriptions may be identically applied with respect to the above, detailed descriptions thereof will be omitted.

An embodiment of filtering being performed on the obtained image 10 will be described below.

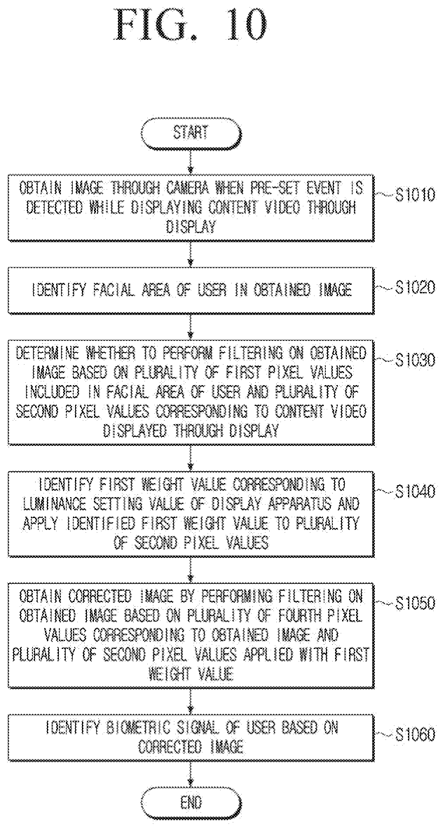

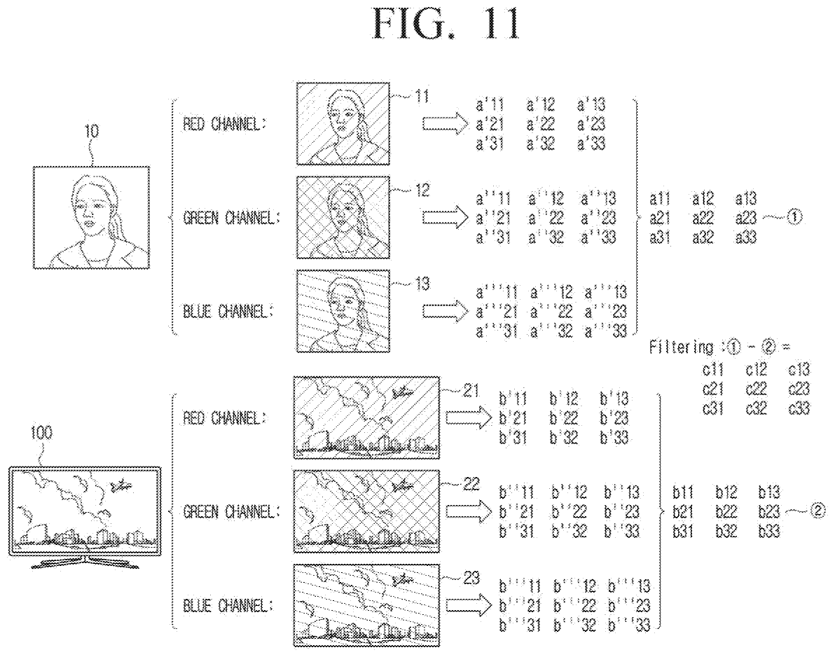

FIG. 10 is a flowchart illustrating a method for performing filtering based on a luminance setting value of the display apparatus 100 according to an embodiment of the disclosure. FIG. 11 is an example diagram illustrating a method for performing filtering based on setting information of the display apparatus 100 according to an embodiment of the disclosure.

The processor 130 according to an embodiment of the disclosure may obtain, based on determining that filtering is to be performed on the obtained image 10, the corrected image by performing filtering on the obtained image 10 based on the plurality of second pixel values (S340).

The filtering may be removing the noise included in the obtained image 10. Here, the noise may be light information emitted from the display 120 while content is displayed through the display 120. In other words, the processor 130 may determine that information was not obtained intact for the surface of the subject (e.g., face of the user) due to light emitted from the display 120 as content is displayed reaching or being reflected from the subject of the camera 110, and perform the filtering process of removing the information of light being emitted in the obtained image 10.

Specifically, the processor 130 may identify, based on determining that filtering is to be performed, the pixel value of the plurality of pixels included in the obtained image 10. For convenience of description below, the pixel included in the obtained image 10 may be referred to as a fourth pixel, and a pixel value of the fourth pixel may be referred to as a fourth pixel value. Meanwhile, the plurality of fourth pixels may include the plurality of second pixels included in the facial area 11 and a plurality of third pixels included in the remaining area 12. The processor 130 may perform filtering on the obtained image 10 based on a plurality of fourth pixel values included in the obtained image 10 and the plurality of second pixel values corresponding to the content. At this time, the content (and the plurality of fourth pixel values of the content) used in performing filtering on the obtained image 10 may be an image frame (and the plurality of fourth pixel values included in the matching image frame) that matches with the obtained image 10 among the plurality of image frames of the content.

The processor 130 may perform filtering by subtracting the plurality of second pixel values (\textcircled{2}) corresponding to the content from the plurality of fourth pixel values (\textcircled{1}) included in the obtained image 10. For example, referring to FIG. 11, the processor 130 may subtract b₁₁ to b₃₃ which are the plurality of second pixel values corresponding to the content from a₁₁ to a₃₃ which are the plurality of fourth pixel values for the obtained image 10. Then, the processor 130 may obtain the corrected image based on c₁₁ to c₃₃—which are the plurality of pixel values (\textcircled{1}-\textcircled{2}) obtained through filtering.

At this time, the processor 130 may perform filtering based on the plurality of fourth pixel values (i.e., a plurality of fourth pixel values a′₁₁ to a′₃₃ of the red channel, a plurality of fourth pixel values a″₁₁ to a″₃₃ of the green channel, and a plurality of fourth pixel values a′″₁₁ to a′″₃₃ of the blue channel) corresponding to the plurality of channels of the obtained image 10 and the plurality of second pixel values (i.e., a plurality of second pixel values b′₁₁ to b′₃₃ of the red channel, a plurality of second pixel values b″₁₁ to b″₃₃ of the green channel, and a plurality of second pixel values b″′₁₁ to b″′₃₃ of the blue channel) corresponding to the plurality of channels of the content. In an example, the processor 130 may subtract the plurality of first pixel values b′₁₁ to b′₃₃ corresponding to the red channel of the content from the plurality of fourth pixel values a′₁₁ to a′₃₃ corresponding to the red channel of the obtained image 10, subtract the plurality of first pixel values b″₁₁ to b″₃₃ corresponding to the green channel of the content from the plurality of fourth pixel values a″₁₁ to a″₃₃ corresponding to the green channel of the obtained image, and subtract the plurality of first pixel values b″′₁₁ to b″′₃₃ corresponding to the blue channel of the content from the plurality of fourth pixel values a″₁₁ to a″₃₃ corresponding to the blue channel of the obtained image 10, and perform filtering on each of the channels. Then, the processor 130 may obtain the corrected image based on the plurality of pixel values c′₁₁ to c′₃₃, c″₁₁ to c″₃₃, and c′″₁₁ to c′″₃₃ obtained for each of the channels (i.e., red channel, green channel, and blue channel) through filtering. In other words, the processor 130 may obtain the corrected image with a value that combined the plurality of pixel values obtained for the plurality of channels.

Meanwhile, b₁₁ to b₃₃, as shown in FIG. 11, may be pixel values obtained by performing normalization on the plurality of second pixel values. In other words, before performing filtering, the processor 130 may perform normalization on the plurality of second pixel values corresponding to the content. In an example, the processor 130 may adjust the plurality of second pixel values within the pre-set range by performing normalization on the plurality of second pixel values for the content. Here, the pre-set range may be set based on the plurality of fourth pixel values included in the obtained image 10. For example, the processor 130 may identify an average value of the plurality of fourth pixel values and then, set the pre-set range based on the identified average value of the plurality of fourth pixel values, and perform normalization on the plurality of second pixel values to have a pixel value within the set pre-set range. Below, the plurality of second pixel values has been collectively described as a pixel value obtained by performing normalization on the plurality of second pixel values.

Meanwhile, in FIG. 11, the plurality of fourth pixel values included in the obtained image 10 and the plurality of second pixel values corresponding to the content have been shown in a 3×3 form, but this is merely for convenience of description, and the form of the plurality of pixel values (i.e., plurality of second pixel values and plurality of fourth pixel values) may have various sizes according to a size of the image, a resolution of the display, and the like.

The processor 130 according to an embodiment of the disclosure may set, based on the information set in the display apparatus 100, a weight value to apply to the plurality of second pixel values corresponding to the content. In an example, the processor 130 may identify a first weight value corresponding to the luminance setting value of the display apparatus 100. Here, the luminance setting value may be a setting value for adjusting the brightness of the display 120. In other words, the processor 130 may identify that intensity of light emitted from the display 120 is great if the luminance setting value is high, and apply to the plurality of second pixel values by setting a weight value that takes into consideration the intensity of light. At this time, the weight value corresponding to the luminance setting value may be determined as a large value to greater the luminance setting value is. In other words, the processor 130 may set the weight value to be proportionate to the luminance setting value. For convenience of description below, the weight value set based on the luminance setting value may be referred to as the first weight value.

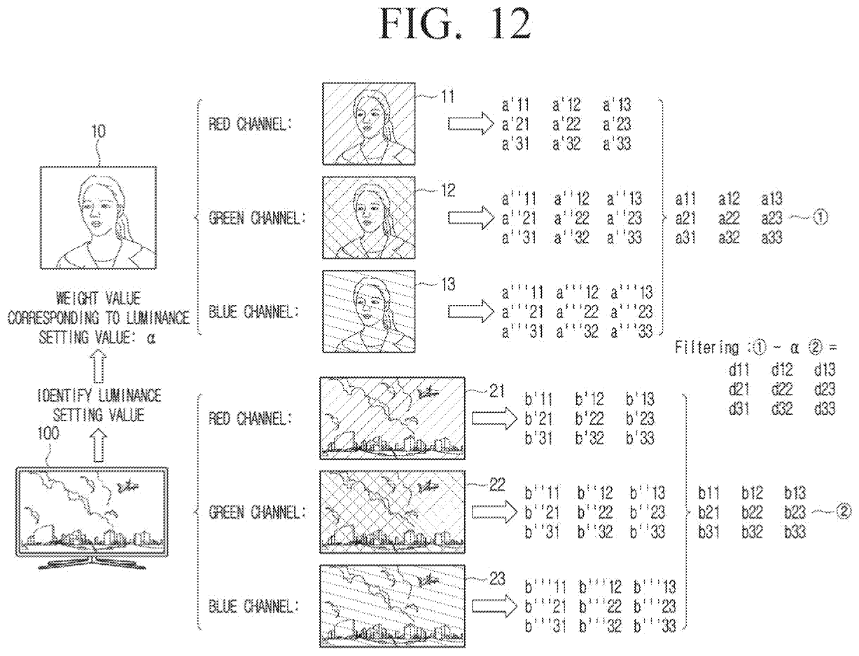

FIG. 12 is an example diagram illustrating performing filtering based on a weight value set based on a luminance setting value according to an embodiment of the disclosure.

Then, the processor 130 may apply the first weight value to the plurality of second pixel values, and perform, based on the plurality of fourth pixel values corresponding to the obtained image 10 and the plurality of second pixel values applied with the first weight value, filtering on the obtained image 10. For example, referring to FIG. 12, the processor 130 may perform filtering by subtracting the plurality of second pixel values b₁₁ to b₃₃ (or pixel value obtained by performing normalization on the plurality of second pixel values) applied with a first weight value a from the plurality of fourth pixel values an to a₃₃. At this time, referring to FIG. 9, the processor 130 may apply the same first weight value to the plurality of second pixel values b′₁₁ to b′₃₃, b″₁₁ to b″₃₃, and b′″₁₁ to b″″₃₃) of the plurality of channels corresponding to the video content.

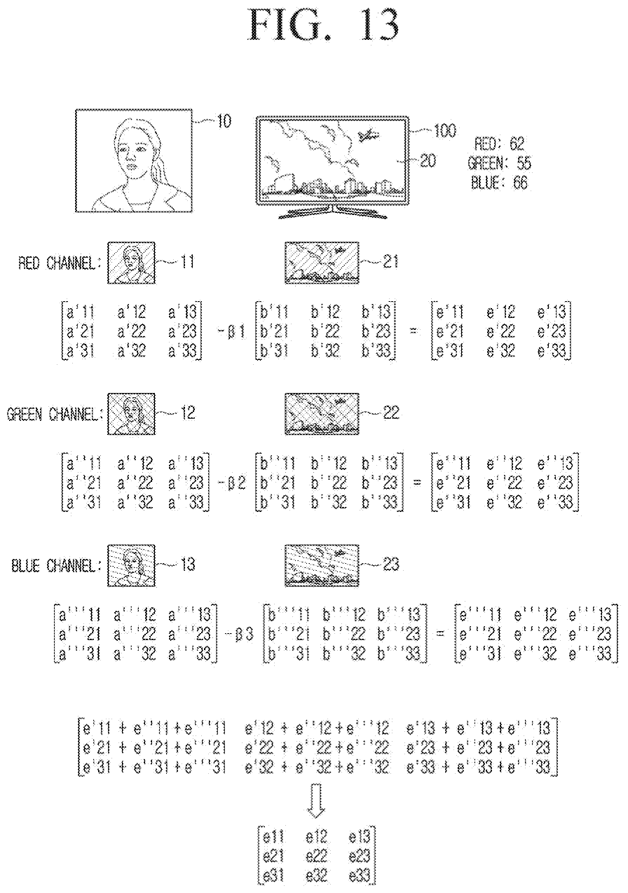

FIG. 13 is an example diagram illustrating performing filtering based on a weight value set based on a hue value set to each red color, green color, and blue color according to one or more embodiments of the disclosure.

Meanwhile, according to one or more embodiments of the disclosure, weight values different from one another may be applied to the plurality of second pixel values that correspond to each of the channels. In this respect, according to an embodiment of the disclosure, the processor 130 may identify the plurality of third pixel values corresponding respectively to the red channel, the blue channel, and the green channel that constitute the obtained image 10 and the plurality of second pixel values corresponding respectively to the red channel, the blue channel, and the green channel that constitute the content. Then, the processor 130 may identify a plurality of weight values corresponding respectively to the red channel, the blue channel, and the green channel that constitute the content based on a hue value set respectively for the red color, the blue color, and the green color of the display apparatus 100. The hue value set respectively for the red color, the blue color, and the green color of the display apparatus 100 may be a color corrected value set in the display apparatus 100. At this time, as the set hue value is greater, a video with enhanced relevant colors may be displayed on the display 120. Conversely, as the set hue value is smaller, a video with subdued relevant colors may be displayed on the display 120. Accordingly, the processor 130 may identify the hue value set for red, green and blue, and identify respective weight values to apply to the plurality of second pixel values of the red channel, the plurality of second pixel values of the green channel, and the plurality of second pixel values of the blue channel based on the identified hue value. For convenience of description below, the weight value set based on the set hue value may be referred to as a second weight value.

Specifically, the processor 130 may respectively set the second weight values to be proportionate to the hue value respectively set for red, green and blue. In addition, the processor 130 may calculate a ratio of the hue values set respectively for red, green and blue, and respectively set the second weight values according to the calculated ratio. At this time, a sum of the plurality of second weight values may be 1.

Referring to FIG. 13, the processor 130 may respectively identify the hue values (red: 62, green: 55, blue: 66) set in the display apparatus 100 for the red, green, and blue colors. Then, the processor 130 may set the second weight values β₁, β₂ and β₃ corresponding respectively to each of the red channel, the green channel, and the blue channel based on the hue values (red: 62, green: 55, blue: 66) set in each of the colors (red, green, and blue).

The processor 130 may apply the identified plurality of second weight values β₁, β₂ and β₃ to the plurality of second pixel values corresponding respectively to the red channel, the blue channel, and the green channel that constitute the content, subtract the plurality of second pixel values corresponding respectively to the red channel, the blue channel, and the green channel that constitute the content applied with the respective second weight values from the plurality of fourth pixel values corresponding respectively to the red channel, the blue channel, and the green channel that constitute the obtained image 10, and perform filtering. In other words, referring to FIG. 13, the processor 130 may apply a second weight value â₁ set corresponding to the red channel to the plurality of second pixel values b′₁₁ to b′₃₃ corresponding to the red channel, apply a second weight value â₂ set corresponding to the green channel to the plurality of second pixel values b″₁₁ to b″₃₃ corresponding to the green channel, and apply a second weight value â₃ set corresponding to the blue channel to the plurality of second pixel values b″′₁₁ to b″′₃₃ corresponding to the blue channel. Then, the processor 130 may perform filtering by subtracting the plurality of second pixel values (i.e., the plurality of second pixel values b′₁₁ to b′₃₃ of the red channel, the plurality of second pixel values b″₁₁ to b″₃₃ of the green channel, and the plurality of fourth pixel values b″′₁₁ to b″′₃₃ of the blue channel) applied with the respective weight values β₁, β₂ and β₃ from the plurality of fourth pixel values (i.e., the plurality of fourth pixel values a′₁₁ to a′₃₃ of the red channel, the plurality of fourth pixel values a″₁₁ to a″₃₃ of the green channel, and the plurality of fourth pixel values a″₁₁ to a′″₃₃ of the blue channel) corresponding to the respective channels. Then, the processor 130 may obtain the corrected image based on the plurality of pixel values c′₁₁ to c′₃₃, c″₁₁ to c″₃₃, and c′″₁₁ to c′″₃₃ obtained for the respective channels (i.e., red channel, green channel, and blue channel) through filtering. In other words, the processor 130 may obtain the corrected image with a value that combined the plurality of pixel values for the plurality of channels.

Meanwhile, the processor 130 may perform filtering by applying the above-described first weight value and the second weight value together to the plurality of second pixel values corresponding to the content. For example, the processor 130 may apply the first weight value a and a second weight value β₁ set based on the hue value of the red color to the plurality of second pixel values corresponding to the red channel, apply the first weight value a and a second weight value β₂ set based on the hue value of the green color to the plurality of second pixel values corresponding to the green channel, and apply the first weight value a and a second weight value β₃ set based on the hue value of the blue color to the plurality of second pixel values corresponding to the blue channel. Then, the processor 130 may perform filtering by subtracting the plurality of second pixel values applied with the first and second weight values from the plurality of first pixel values corresponding to the respective channels.

Meanwhile, the processor 130 may set the weight value to be applied to the plurality of second pixel values corresponding to the content based on the contrast ratio of the facial area 11 of the user compared to the remaining area 12. If the contrast ratio is small, the processor 130 may identify that the difference in brightness of the facial area 11 of the user and the remaining area 12 is great, and identify that the facial area 11 of the user is affected by the light emitted from the display 120. Specifically, the processor 130 may identify that a size of intensity of light or an amount of light emitted from the display is great the smaller the contrast ratio is. Accordingly, the processor 130 may set the weight value to the plurality of second pixel values corresponding to the content based on the contrast ratio. At this time, the processor 130 may determine for the weight value to be inversely proportionate to the contrast ratio. For convenience of description below, the weight value set based on the brightness ratio of the facial area 11 compared to the remaining area 12 may be referred to as a third weight value.

According to an embodiment of the disclosure, the processor 130 may identify the remaining area 12 other than the facial area 11 of the user in the obtained image 10, and identify the contrast ratio of the facial area 11 of the user compared to the remaining area 12 based on the plurality of third pixel values and the plurality of first pixel values included in the identified remaining area 12. In this respect, because the above-described descriptions in FIG. 4 may be identically applied, detailed descriptions thereof will be omitted.

Then, the processor 130 may identify the third weight value corresponding to the identified contrast ratio, apply the identified third weight value to the plurality of second pixel values, and perform filtering on the obtained image 10 based on the plurality of fourth pixel values corresponding to the obtained image 10 and the plurality of fourth pixel values applied with the third weight value.

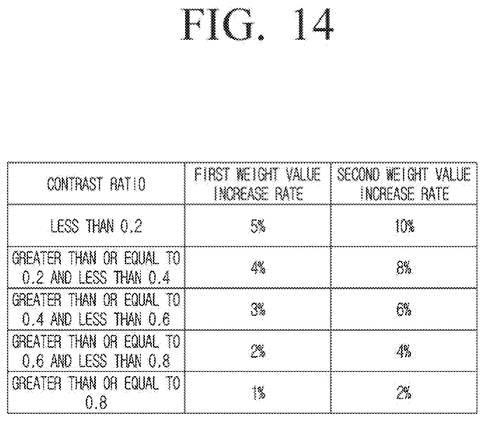

FIG. 14 is an example diagram illustrating adjusting of first and second weight values based on a contrast ratio according to an embodiment of the disclosure.

Meanwhile, the processor 130 may adjust the first and second weight values based on the contrast ratio of the facial area 11 compared to the identified remaining area 12. Referring to FIG. 14, the processor 130 may increase the first and second weight values according to the contrast ratio. In other words, the processor 130 may identify that the intensity of light emitted from the display 120 is great or the amount of light as great the smaller the contrast ratio is, and increase the first and second weight values. Meanwhile, an increase rate shown in FIG. 14 is merely one example, and a range of the contrast ratio and increase rates of the first and second weight values according to the range of the contrast ratio may be variously set.

The processor 130 according to an embodiment of the disclosure may identify the biometric signal of the user based on the corrected image (S350). Here, the biometric signal may include a pulse signal (e.g., a plethysmogram (PPG) signal), a blood pressure signal, a heart-rate signal, a Ballistocardiogram (BCG), and the like of the user. The processor 130 may identify biometric information of the user (e.g., pulse rate, blood pressure, heart rate, etc.) based on the identified biometric signal.

The processor 130 may detect a change in skin color of the face of the user based on a plurality of corrected images. Specifically, the processor 130 may obtain the plurality of corrected images based on determining that filtering is to be performed. The processor 130 may obtain, based on determining that filtering is to be performed, the plurality of corrected images by repeatedly obtaining an image of the user through the camera 10 thereafter and performing filtering on the obtained plurality of images. The processor 130 may detect the facial area 11 of the user in the plurality of corrected images obtained according to time, and identify the plurality of pixel values included in the facial area 11 of the user. Here, the pixel value identified in the corrected image may be a pixel value calculated through filtering. Then, the processor 130 may calculate an average value of the plurality of pixel values included in the facial area 11 of the respective corrected images, and identify the change in skin color of the face of the user based on the calculated average value. Specifically, the processor 130 may obtain a video signal about the plurality of corrected images based on the calculated average value. The obtained video signal may be time series data. The processor 130 may identify the change in skin color of the face of the user based on the obtained video signal.

Then, the processor 130 may detect periodicity on the changes in skin color of the face of the user, and identify the biometric signal of the user based on the detected periodicity. For example, the processor 130 may identify a frequency component included in the obtained video signal. The processor 130 may identify the frequency component included in the video signal through the Fast Fourier Transform (FFT) of the obtained video signal, and detect pulse signals (pulse wave signals) associated with the pulse of the user from the identified frequency component. Then, the processor 130 may determine the biometric information (e.g., pulse rate, blood pressure, heart rate, etc.) of the user based on the detected pulse signals (pulse signal associated with pulse or pulse wave signal). Meanwhile, the processor 130 may display the biometric information of the user on the display 120 and provide to the user. In addition, the processor 130 may analyze the health state of the user based on the biometric information and display information about the health state of the user on the display 120.

Meanwhile, the processor 130 according to an embodiment of the disclosure may identify a moving average value for a signal to noise ratio (SNR) of the biometric signal based on the obtained plurality of corrected images, and stop obtaining of the image through the camera 110 based on the identified moving average value being identified as less than the pre-set third value for the pre-set time.

The processor 130 may calculate the signal-to-noise ratio of the biometric signal based on Equation 1 below.

$$( Equation 1 )$$

$$SNR ( signal - to - noise ratio of biometric signal ) = 10 {log}_{10} ( \frac{{\sum }_{f = f 1}^{{f}_{2}} {( \vec{{U}_{t} ( f )} \vec{\hat{S} ( f} )}^{2}}{{\sum }_{f = f 1}^{{f}_{2}} {( 1 - \vec{{U}_{t} ( f )} \vec{\hat{S} ( f} )}^{2}} )$$

(Here, S (f) may represent the biometric signal, f may represent the frequency, Ut (f) may represent a double step function, f1 may represent 0.5 Hz, and f2 may represent 3.5 Hz.)

Specifically, the processor 130 may calculate the signal-to-noise ratio of the identified biometric signal based on the biometric signal being identified based on the obtained plurality of corrected images. For example, the processor 130 may detect the pulse signal (pulse wave signal) associated with the pulse based on the obtained plurality of corrected images, and identify the signal-to-noise ratio of the pulse signal (pulse wave signal) associated with the detected pulse.

The processor 130 may identify the moving average value of the signal-to-noise ratio. Specifically, the processor 130 may identify an average value of the signal-to-noise ratio for every pre-set time. At this time, the processor 130 may stop the obtaining of the image through the camera 110 based on identifying that the signal-to-noise ratio for the pre-set time is less than the pre-set value. The processor 130 may determine that an accurate biometric signal and biometric information about the user is not obtainable through the obtained image 10 based on the moving average value of the signal-to-noise ratio being identified as less than the pre-set value, and stop the obtaining of the image through the camera 110. Then, the processor 130 may obtain, when the pre-set event is detected again thereafter, the biometric signal and biometric information about the user by obtaining a plurality of images of the user through the camera 110 again.

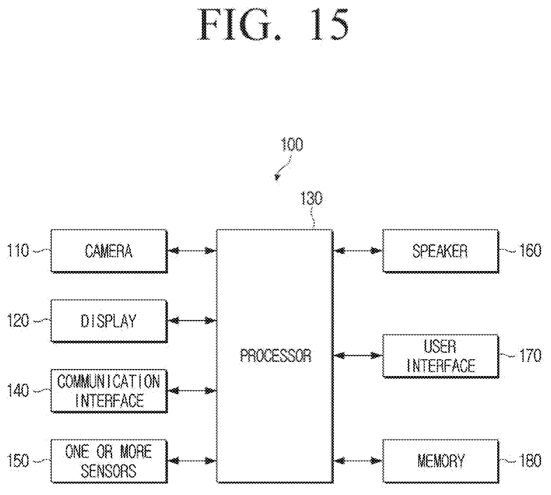

FIG. 15 is a detailed block diagram of the display apparatus 100 according to an embodiment of the disclosure.

Referring to FIG. 15, the display apparatus 100 may include the camera 110, the display 120, a communication interface 140, one or more sensors 150, a speaker 160, a user interface 170, memory, and the processor 130. Detailed descriptions of configurations that overlap with the configurations shown in FIG. 2 among the configurations shown in FIG. 15 will be omitted.

The communication interface 140 may transmit or receive content of various types. In an example, the processor 130 may receive content through the communication interface 140, or transmit the biometric information of the user to the user terminal. In addition, the processor 130 may receive a plurality of images through the communication interface 140. In other words, the processor 130 may obtain the plurality of images of the user from the external camera 110 disposed adjacently to the display apparatus 100 and not the camera 110.

The communication interface 140 may transmit or receive signals in a streaming or download method from an external apparatus (e.g., user terminal), an external storage medium (e.g., USB memory), or an external server (e.g., WEBHARD) through communication methods such as, for example, and without limitation, an AP based Wi-Fi (e.g., wireless LAN network), Bluetooth, ZigBee, a wired/wireless local area network (LAN), a wide area network (WAN), Ethernet, IEEE 1394, a high-definition multimedia interface (HDMI), a universal serial bus (USB), a mobile high-definition link (MHL), Audio Engineering Society/European Broadcasting Union (AES/EBU), Optical, Coaxial, or the like.

Meanwhile, the display apparatus 100 may include one or more sensors 150. The one or more sensors 150 may include a sensor (e.g., LiDAR sensor, ToF sensor, etc.) for detecting an object in the surrounding of the display apparatus 100. In addition thereto, the one or more sensors 150 may further include at least one among a gesture sensor, a gyro sensor, a barometric sensor, a magnetic sensor, an acceleration sensor, a grip sensor, a proximity sensor, a color sensor (e.g., red, green, blue (RGB) sensor), a biometric sensor, a temperature/humidity sensor, an ambient light sensor, or an ultra violet (UV) sensor.

The speaker 160 may output sound signals to the outside of the display apparatus 100. The speaker 160 may output multi-media playbacks, recording playbacks, various notification sounds, voice messages, and the like. The display apparatus 100 may include an audio output apparatus such as a speaker 160, but may include an output apparatus such as an audio output terminal. Specifically, the speaker 160 may provide obtained information, information processed manufactured based on the obtained information, a response result or an operation result to a user voice, and the like in voice form. In an example, the processor 130 may output, based on the health state of the user identified based on the biometric signal being identified as in danger, a warning sound or a voice message through the speaker 160.

The user interface 180 may be a configuration used by the display apparatus 100 in performing interface with the user, and the processor 130 may receive various information such as control information of the display apparatus 100 through the user interface 170. Specifically, setting information (e.g., luminance setting value or hue value) about the display apparatus 100 may be received through the user interface 170. Meanwhile, the user interface 170 may include at least one among a touch sensor, a motion sensor, a button, a jog dial, a switch, and a microphone, but is not limited thereto.

The memory 180 store data necessary for the various embodiments of the disclosure. In an example, the memory 180 may be stored with video content that is displayed in the display apparatus 100.

The memory 180 may be implemented in a form of a memory embedded in the display apparatus 100 according to data storage use, or implemented in a form of memory attachable to or detachable from the display apparatus 100. For example, data for driving the display apparatus 100 may be stored in the memory embedded in the display apparatus, and data for an expansion function of the display apparatus 100 may be stored in the memory attachable to and detachable from the display apparatus 100.

Meanwhile, the memory embedded in the display apparatus 100 may be implemented as at least one of a volatile memory (e.g., dynamic RAM (DRAM), static RAM (SRAM), synchronous dynamic RAM (SDRAM), etc.) or a non-volatile memory (e.g., one time programmable ROM (OTPROM), programmable ROM (PROM), erasable and programmable ROM (EPROM), electrically erasable and programmable ROM (EEPROM), mask ROM, flash ROM, flash memory (e.g., NAND flash, NOR flash, etc), hard drive, or solid state drive (SSD)).

In addition, the memory attachable to or detachable from the display apparatus 100 may be implemented in a form such as, for example, and without limitation, a memory card (e.g., compact flash (CF), secure digital (SD), micro secure digital (Micro-SD), mini secure digital (Mini-SD), extreme digital (xD), multi-media card (MMC), etc.), an external memory connectable to a USB port (e.g., USB memory), or the like.

According to an example, the memory 180 may store information about a plurality of neural network (or artificial intelligence) models. In an example, the memory 180 may store a neural network model trained to identify a biometric signal of the user based on the obtained corrected image. In other words, by inputting the plurality of corrected images in the neural network model, the processor 130 may obtain a biometric signal about the user.

Here, storing information about the neural network model may mean storing various information associated with an operation of the neural network model such as, for example, and without limitation, information about at least one layer included in the neural network model, a parameter used in each of the at least one layer, information about vias, and the like. However, information about the neural network model may be stored in an internal memory of the processor 130 according to an implementation form of the processor 130. For example, if the processor 130 is implemented with a dedicated hardware, information about the neural network model may be stored in the internal memory of the processor 130.

Meanwhile, methods according to the various embodiments of the disclosure described above may be implemented in an application form installable in a display apparatus of the related art. Alternatively, methods according to the various embodiments of the disclosure described above may be performed using a deep learning-based trained neural network (or deep trained neural network), in other words, a trained network model. In addition, the methods according to the various embodiments of the disclosure described above may be implemented with only a software upgrade, or a hardware upgrade for the display apparatus of the related art. In addition, the various embodiments of the disclosure described above may be performed through an embedded server provided in the display apparatus, or an external server of the display apparatus.

Meanwhile, according to an embodiment of the disclosure, the various embodiments described above may be implemented with software including instructions stored in a machine-readable storage media (e.g., computer). The machine may call the stored instructions from the storage media, and as an apparatus operable according to the called instructions, may include a display apparatus (e.g., display apparatus (A)) according to the above-mentioned embodiments. Based on a command being executed by the processor, the processor may directly or using other elements under the control of the processor perform a function relevant to the command. The command may include a code generated by a compiler or executed by an interpreter. The machine-readable storage media may be provided in a form of a non-transitory storage medium. Herein, ‘non-transitory’ merely means that the storage medium is tangible and does not include a signal, and the term does not differentiate data being semi-permanently stored or being temporarily stored in the storage medium.

In addition, according to an embodiment, a method according to the various embodiments described above may be provided included a computer program product. The computer program product may be exchanged between a seller and a purchaser as a commodity. The computer program product may be distributed in the form of the machine-readable storage medium (e.g., a compact disc read only memory (CD-ROM)), or distributed online through an application store (e.g., PLAYSTORE™). In the case of online distribution, at least a portion of the computer program product may be stored at least temporarily in the storage medium such as a server of a manufacturer, a server of an application store, or memory of a relay server, or temporarily generated.

In addition, each of the elements (e.g., module or program) according to the various embodiments described above may be configured as a single entity or a plurality of entities, and a portion of sub-elements of the above-mentioned relevant sub-elements may be omitted or other sub-elements may be further included in the various embodiments. Alternatively or additionally, a portion of the elements (e.g., modules or programs) may be integrated into one entity to perform the same or similar function performed by the respective relevant elements prior to integration. Operations performed by a module, a program, or other element, in accordance with the various embodiments, may be executed sequentially, in parallel, repetitively, or in a heuristically manner, or at least a portion of the operations may be performed in a different order, omitted, or a different operation may be added.

While example embodiments of the disclosure have been shown and described above, it will be understood that disclosure is not limited to the above-described specific embodiments, and that various changes in form and details may be made by those of ordinary skill in the art to which this disclosure pertains without departing from the true spirit and full scope of the disclosure, including the appended claims and their equivalents.

What is claimed is:

1. A display apparatus, comprising:
a camera;
a display;
memory storing one or more instructions; and
at least one processor configured to execute the one or more instructions,
wherein the one or more instructions, when executed by the at least one processor individually or collectively, cause the display apparatus to:
based on an occurrence of a pre-set event, obtain an image through the camera while content is being output through the display,
determine, based on a plurality of first pixel values corresponding to a facial area of a user identified in the obtained image and a plurality of second pixel values corresponding to the content, whether to perform filtering on the obtained image,
based on a determination that filtering is to be performed on the obtained image, obtain a corrected image by performing filtering on the obtained image based on the plurality of second pixel values, and
identify a biometric signal of the user based on the corrected image.

2. The display apparatus of claim 1, wherein the one or more instructions, when executed by the at least one processor individually or collectively, cause the display apparatus to:
determine to perform filtering on the obtained image based on a correlation degree between the plurality of first pixel values and the plurality of second pixel values being greater than or equal to a pre-set first value.

3. The display apparatus of claim 1, wherein the one or more instructions, when executed by the at least one processor individually or collectively, cause the display apparatus to:
identify, based on the plurality of first pixel values corresponding to the facial area of the user and a plurality of third pixel values corresponding to a remaining area other than the facial area of the user identified from the obtained image, a contrast ratio between the facial area and the remaining area, and
determine to perform filtering on the obtained image based on the identified contrast ratio being greater than or equal to a pre-set second value.

4. The display apparatus of claim 1, wherein the one or more instructions, when executed by the at least one processor individually or collectively, cause the display apparatus to:
determine to perform filtering on the obtained image based on an average value of the plurality of second pixel values being greater than or equal to a pre-set third value, the plurality of second pixel values corresponding to a green channel among a plurality of channels of the content.

5. The display apparatus of claim 1, wherein the one or more instructions, when executed by the at least one processor individually or collectively, cause the display apparatus to:
for each image frame of a plurality of image frames of the content, identify an average value of the plurality of second pixel values corresponding to a green channel of the image frame,
identify, based on each average value corresponding to each image frame, a frequency corresponding to a green channel of the content, and
determine to perform filtering on the obtained image based on the identified frequency being within a pre-set range and each average value of each frame of the plurality of image frames of the content being greater than or equal to a pre-set third value.

6. The display apparatus of claim 1, wherein the one or more instructions, when executed by the at least one processor individually or collectively, cause the display apparatus to:
apply a first weight value corresponding to a luminance setting value of the display apparatus to the plurality of second pixel values, and
perform, based on a plurality of fourth pixel values corresponding to the obtained image and the plurality of second pixel values applied with the first weight value, filtering on the obtained image.

7. The display apparatus of claim 1, wherein the one or more instructions, when executed by the at least one processor individually or collectively, cause the display apparatus to:
obtain, based on the plurality of first pixel values corresponding to the facial area of the user and a plurality of third pixel values corresponding to a remaining area other than the facial area of the user identified from the obtained image, a contrast ratio between the facial area and the remaining area,
apply a second weight value corresponding to the obtained contrast ratio to the plurality of second pixel values, and
perform, based on a plurality of fourth pixel values corresponding to the obtained image and the plurality of second pixel values applied with the second weight value, filtering on the obtained image.

8. The display apparatus of claim 1, wherein the one or more instructions, when executed by the at least one processor individually or collectively, cause the display apparatus to:
identify a plurality of third pixel values corresponding respectively to a red channel, a blue channel, and a green channel of the obtained image,
identify, based on hue values respectively set for a red color, a blue color, and a green color of the display apparatus, a plurality of third weight values corresponding respectively to a red channel, a blue channel, and a green channel of the content,
apply the identified plurality of third weight values to the plurality of second pixel values, the plurality of second pixel values corresponding respectively to the red channel, the blue channel, and the green channel of the content,
obtain a plurality of fifth pixel values corresponding respectively to the red channel, the blue channel, and the green channel of the corrected image by subtracting the plurality of second pixel values applied with the plurality of third weight values from a plurality of fourth pixel values, the plurality of fourth pixel values corresponding respectively to the red channel, the blue channel, and the green channel of the obtained image, and
obtain the corrected image based on the plurality of fifth pixel values.

9. The display apparatus of claim 1, wherein the one or more instructions, when executed by the at least one processor individually or collectively, cause the display apparatus to:
obtain a plurality of images through the camera,
based on the determination that filtering is to be performed on the obtained image, obtain a plurality of corrected images by performing filtering on the plurality of images obtained through the camera,
obtain a moving average value of a signal-to-noise ratio of the biometric signal based on the obtained plurality of corrected images, and
stop obtaining the plurality of images based on the moving average value being less than a pre-set third value for a pre-set time.

10. A method for controlling a display apparatus, the method comprising:
based on an occurrence of a pre-set event, obtaining an image through a camera of the display apparatus while content is being output through a display of the display apparatus;
determining, based on a plurality of first pixel values corresponding to a facial area of a user identified in the obtained image and a plurality of second pixel values corresponding to the content displayed through the display, whether to perform filtering on the obtained image;
based on determining that filtering is to be performed on the obtained image, obtaining a corrected image by performing filtering on the obtained image based on the plurality of second pixel values; and
identifying a biometric signal of the user based on the corrected image.

11. The method of claim 10, wherein
the determining whether to perform filtering on the obtained image comprises:
determining to perform filtering on the obtained image based on a correlation degree between the plurality of first pixel values and the plurality of second pixel values being greater than or equal to a pre-set first value.

12. The method of claim 10, wherein
the determining that filtering is to be performed on the obtained image comprises:
identifying, based on the plurality of first pixel values corresponding to the facial area of the user and a plurality of third pixel values corresponding to a remaining area other than the facial area of the user identified from the obtained image, a contrast ratio between the facial area and the remaining area; and
determining to perform filtering on the obtained image based on the identified contrast ratio being greater than or equal to a pre-set second value.

13. The method of claim 10, wherein
the determining whether to perform filtering on the obtained image comprises:
determining to perform filtering on the obtained image based on an average value of the plurality of second pixel values being greater than or equal to a pre-set third value, the plurality of second pixel values corresponding to a green channel among a plurality of channels of the content.

14. The method of claim 10, further comprising:
for each image frame of a plurality of image frames of the content, identifying an average value of the plurality of second pixel values corresponding the a green channel of the image frame;
identifying, based on each average value corresponding to each image frame, a frequency corresponding to a green channel of the content; and
determining to perform filtering on the obtained image based on the identified frequency being within a pre-set range and each average value of each frame of the plurality of image frames of the content being greater than or equal to a pre-set third value.

15. The method of claim 10, wherein
the obtaining the corrected image comprises:
applying a first weight value corresponding to a luminance setting value of the display apparatus to the plurality of second pixel values; and
performing, based on a plurality of fourth pixel values corresponding to the obtained image and the plurality of second pixel values applied with the first weight value, filtering on the obtained image.

16. The method of claim 10, wherein the obtaining the corrected image comprises:
obtaining, based on the plurality of first pixel values corresponding to the facial area of the user and a plurality of third pixel values corresponding to a remaining area other than the facial area of the user identified from the obtained image, a contrast ratio between the facial area and the remaining area,
applying a second weight value corresponding to the obtained contrast ratio to the plurality of second pixel values, and
performing, based on a plurality of fourth pixel values corresponding to the obtained image and the plurality of second pixel values applied with the second weight value, filtering on the obtained image.

17. The method of claim 10, wherein the obtaining the corrected image comprises:
identifying a plurality of third pixel values corresponding respectively to a red channel, a blue channel, and a green channel of the obtained image,
identifying, based on hue values respectively set for a red color, a blue color, and a green color of the display apparatus, a plurality of third weight values corresponding respectively to a red channel, a blue channel, and a green channel of the content,
applying the identified plurality of third weight values to the plurality of second pixel values, the plurality of second pixel values corresponding respectively to the red channel, the blue channel, and the green channel of the content,
obtaining a plurality of fifth pixel values corresponding respectively to the red channel, the blue channel, and the green channel of the corrected image by subtracting the plurality of second pixel values applied with the plurality of third weight values from a plurality of fourth pixel values, the plurality of fourth pixel values corresponding respectively to the red channel, the blue channel, and the green channel of the obtained image, and
obtaining the corrected image based on the plurality of fifth pixel values.

18. The method of claim 10, further comprising:
obtaining a plurality of images through the camera,
based on determining that filtering is to be performed on the obtained image, obtaining a plurality of corrected images by performing filtering on the plurality of images obtained through the camera,
obtaining a moving average value of a signal-to-noise ratio of the biometric signal based on the obtained plurality of corrected images, and
stopping the obtaining the plurality of images based on the moving average value being less than a pre-set third value for a pre-set time.

19. The display apparatus of claim 1, wherein the one or more instructions, when executed by the at least one processor individually or collectively, cause the display apparatus to:
identify, based on the plurality of first pixel values corresponding to the facial area of the user and a plurality of third pixel values corresponding to a remaining area other than the facial area of the user identified from the obtained image, a contrast ratio between the facial area and the remaining area,
identify an average value of the plurality of second pixel values, the plurality of second pixel values corresponding to a green channel among a plurality of channels of the content,
identify whether a correlation degree between the plurality of first pixel values and the plurality of second pixel values is greater than or equal to a pre-set first value,
based on the correlation degree between the plurality of first pixel values and the plurality of second pixel values being greater than or equal to the pre-set first value, identify whether the contrast ratio is greater than or equal to a pre-set second value,
based on the contrast ratio being greater than or equal to the pre-set second value, identify whether the average value of the plurality of second pixel values is greater than or equal to a pre-set third value, and
based on the average value of the plurality of second pixel values being greater than or equal to the pre-set third value, determine to perform filtering on the obtained image.

20. A non-transitory computer-readable medium storing instructions that, when executed by one or more processors of a display apparatus, cause the one or more processors to:
based on an occurrence of a pre-set event, obtain an image through a camera of the display apparatus while content is being output through a display of the display apparatus;
determine, based on a plurality of first pixel values corresponding to a facial area of a user identified in the obtained image and a plurality of second pixel values corresponding to the content, whether to perform filtering on the obtained image;
based on a determination that filtering is to be performed on the obtained image, obtain a corrected image by performing filtering on the obtained image based on the plurality of second pixel values; and
identify a biometric signal of the user based on the corrected image.
